(19) United States
(12) Patent Application Publication
(10) Pub. No.: US 2009/0181075 A1
(43) Pub. Date: Jul. 16, 2009

(54) DROSPIRENONE CONTAINING TRANSDERMAL DRUG DELIVERY DEVICES AND METHODS OF DELIVERY THEREOF

(76) Inventors: Ryan D. Gordon, Saint Paul, MN (US); Peter M. Seiler, Saint Paul, MN (US); Donald T. Landin, Saint Paul, MN (US); Stefan Bracht, Berlin (DE); Wolfgang Eder, Montville (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/995,767

(22) PCT Filed: Jul. 13, 2006
(86) PCT No.: PCT/US2006/027458

§ 371 Date: Jan. 13, 2009

Related U.S. Application Data
(60) Provisional application No. 60/699,696, filed on Jul. 15, 2005.
(60) Provisional application No. 60/730,296, filed on Oct. 26, 2005.
(60) Provisional application No. 60/747,217, filed on May 15, 2006.

(51) Int. Cl. A61K 31/585; A61K 9/70

(52) U.S. Cl. 424/449; 514/173; 514/170

(57) ABSTRACT

A transdermal drug delivery device comprising an adhesive matrix, an effective amount of drospirenone, and an oligomeric adjuvant selected from an oligolactic acid, oligolactic acid derivatives, or mixtures thereof. Also, a transdermal drug delivery device comprising a backing film and an adhesive matrix which comprises an effective amount of drospirenone, a solubilizing agent, and a permeation enhancer selected from the group consisting of alkyl lactates, carboxylic acids, alkyl esters of fatty acids, and mixtures thereof.

### CROSS-REFERENCE TO RELATED APPLICATIONS

The present application claims priority to U.S. Provisional Application Ser. No. 60/699,696, filed on Jul. 15, 2005, U.S. Provisional Application Ser. No. 60/730,296, filed on Oct. 26, 2005, and U.S. Provisional Application Ser. No. 60/747,217, filed on May 15, 2006, which are incorporated herein in their entirety.

The claimed invention was made by or on behalf of parties to a joint research agreement that was in effect on or before the date the claimed invention was made and the claimed invention was made as a result of activities undertaken within the scope of the joint research agreement. The names of the parties to the joint research agreement are Schering A. G. of Berlin, Germany and 3M Company of St. Paul, Minn.

The present invention relates to a transdermal drug delivery device and methods of transdermal drug delivery. In particular, the present invention relates to a device for delivering drospirenone.

### BACKGROUND OF THE INVENTION

Drospirenone (6β,7β,15β,16β-dimethylene-3-oxo-17α-pregn-4-ene-21,17-carbolactone) is known from U.S. Pat. No. 4,129,564 in which its use as a diuretic compound is disclosed.

U.S. Pat. No. 5,569,652 discloses the use of drospirenone for the treatment of hormonal irregularities during premenopause (menstruation stabilization), for hormonal substitution therapy during menopause, for treatment of androgen-induced disorders and/or for contraception.

U.S. Pat. No. 6,787,531 discloses a pharmaceutical composition comprising drospirenone and ethinyl estradiol, a method of providing dissolution of drospirenone, methods of inhibiting ovulation by administration of drospirenone and the use of drospirenone and ethinyl estradiol for inhibiting ovulation.

Oral contraceptives containing a combination of a progestin and an estrogen have been used since the 1960's. Transdermal contraceptive compositions were proposed in the late 1980's and a transdermal contraceptive product containing ethinyl estradiol and norelgestromin, Ortho Evra™, was approved for sale in the United States in 2001.

U.S. Pat. No. 4,816,258 discloses a transdermal delivery system for administering ethinyl estradiol and levonorgestrel in combination, utilizing a polymer matrix having the drug formulation along with a permeation enhancer dispersed throughout.

U.S. Pat. No. 5,252,334 discloses a matrix, formed of a skin-adhesive acrylate copolymer, which attains high rates of drug delivery without the addition of drug delivery rate enhancers. In preferred embodiments the matrix is used to administer steroids, in particular estradiol.

U.S. Pat. No. 6,071,531 discloses transdermal patch compositions to administer 17-deacetyl norgestimate alone or in combination with an estrogen such as ethinyl estradiol to women.

### SUMMARY OF THE INVENTION

The present invention provides drug delivery devices for the transdermal delivery of drospirenone. In particular, the present invention provides devices capable of solubilizing and/or delivering relatively large amounts of drospirenone to a patient over an extended period of time.

In one aspect, the present invention is a transdermal drug delivery device comprising an adhesive matrix, an effective amount of drospirenone, and an oligomeric adjuvant selected from an oligolactic acid, oligolactic acid derivatives, or mixtures thereof.

In a second aspect, the present invention is a transdermal drug delivery device comprising a backing film and an adhesive matrix which comprises an effective amount of drospirenone, a solubilizing agent, and a permeation enhancer selected from the group consisting of alkyl lactates, hydroxyacids, alkyl esters of fatty acids, and mixtures thereof. In one embodiment the alkyl ester of a fatty acid is methyl laurate. In one embodiment the solubilizing agent is an aromatic solubilizing agent.

In a third aspect, the present invention is a transdermal drug delivery device comprising an adhesive matrix comprising an effective amount of drospirenone and a permeation enhancer. The concentration of drospirenone in the matrix is above about 5% by weight of the total weight of the matrix and the matrix is substantially free of undissolved drospirenone.

In a fourth aspect, the present invention is a method of providing contraception to a human female comprising the following steps. Providing a transdermal drug delivery system having a total surface area of no more than 25 cm² and comprising a pressure sensitive adhesive matrix having at least about 20 mg of dissolved drospirenone. Placing the delivery system in a delivering relationship to the skin of a human female. Delivering an amount of about 1 to 3 mg/day of drospirenone to the female for a period of about 7 days. In a preferred embodiment, the device also comprises ethinyl estradiol and ethinyl estradiol is delivered in an amount of about 0.01 to 0.03 mg/day to the female for a period of about 7 days.

The above summary of the present invention is not intended to describe each disclosed embodiment or every implementation of the present invention. The detailed description that follows more particularly exemplifies illustrative embodiments, but should not be construed to unduly limit this invention.

### DETAILED DESCRIPTION

In one embodiment, the present invention is a transdermal drug delivery device comprising an adhesive matrix, drospirenone, and an oligomeric adjuvant. Adhesives of the present invention generally comprise a polymer. In some embodiments, the polymer is selected from the group consisting of acrylates, natural rubbers, synthetic rubbers, such as polyisobutylenes, polyisoprenes, styrenic block copolymers, polyvinylethers, silicone polymers, polyurethanes, polyurethane-ureas, and combinations thereof. The adhesive is preferably suitable for use as a skin-contacting adhesive. In one embodiment, the skin-contacting adhesive may comprise a pressure-sensitive adhesive.

Preferred pressure-sensitive adhesives for use in devices of the invention include acrylates, polyisobutylenes, silicone polymers, and mixtures thereof. Examples of useful polyisobutylene pressure-sensitive adhesives are described in U.S. Pat. No. 5,985,317 (Venkateshwaran et al.), the disclosure of which is incorporated herein by reference in its entirety for all purposes. Examples of useful acrylate and silicone polymer pressure-sensitive adhesives, and mixtures thereof, are described in U.S. Pat. No. 5,474,783 (Miranda), the disclosure of which is incorporated herein by reference in its entirety for all purposes. The skin-contacting adhesive may optionally contain other additives, for example, tackifiers, plasticizers, anti-oxidants, colorants, crystallization inhibitors, and the like.

Acrylate polymers and copolymers are particularly preferred pressure-sensitive adhesives. Examples of suitable monomers for use in acrylate copolymers include alkyl acrylates, such as isooctyl, 2-ethylhexyl, n-butyl, ethyl, methyl, and dimethylhexyl, and alkyl methacrylates, such as lauryl, isodecyl, and tridecyl. Isooctyl and 2-ethylhexyl are particularly preferred alkyl acrylate monomers. Monomers containing functional groups, such as carboxylic acid, hydroxy, amide, and amino may also be incorporated into an acrylate copolymer. Examples of suitable monomers containing functional groups include acrylic acid, hydroxyalkyl acrylates containing 2 to 4 carbon atoms in the hydroxyalkyl group, acrylamide, N-vinyl-2-pyrrolidone, vinyl acetate, and alkoxyethyl acrylates. Copolymers containing acrylamide functional groups are particularly preferred. The amount of acrylamide functional group typically ranges from about 1% to about 15%, often from about 5 to about 12%, and sometimes from about 8 to about 11%, by weight of the total weight of copolymer.

Acrylate copolymers may optionally further comprise a substantially linear macromonomer copolymerizable with the other monomers. Suitable macromonomers include polymethylmethacrylate, styrene/acrylonitrile copolymer, polyether, and polystyrene macromonomers. Examples of useful macromonomers and their preparation are described in U.S. Pat. No. 4,693,776 (Krampe et al.), the disclosure of which is incorporated herein by reference in its entirety for all purposes.

In one aspect, the adhesive matrix of the present invention comprises an oligomeric adjuvant selected from oligolactic acid, oligolactic acid derivatives, and mixtures thereof. The oligomeric adjuvant is preferably oligolactic acid.

Oligolactic acid is an oligomer chain derived from a precursor hydroxy-acid that is lactic acid. As the terminology is used herein, a chain “derived from” lactic acid need not be prepared from lactic acid, but rather this terminology is used to designate chains having a structure that could formally be obtained by condensation of lactic acid. The structure of oligolactic acid may be generally represented by formula I:

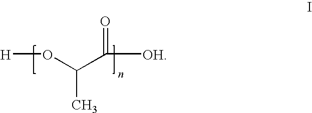

Oligolactic acid derivatives are oligomeric chains derived from oligolactic acid. Again, as the terminology is used herein, chains “derived from” oligolactic acid need not be prepared from oligolactic acid, but rather this terminology is used to designate chains having a structure that could formally be obtained by derivatizing one or both terminal groups of an oligolactic acid. That is, an oligolactic acid derivative comprises oligolactic acid where one or both of the end groups (i.e., the terminal hydrogen and/or hydroxy) is replaced by an R group.

In one embodiment, an oligolactic acid or an oligolactic acid derivative is a compound of the formula II:

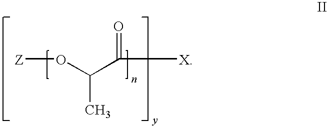

Each oligolactic acid derivative chain is capped on one end by an end group, Z, wherein Z is hydrogen or —C(O)R₁. Each R₁ is independently selected from a linear, branched, or cyclic alkyl, alkoxy, or aryl with 1 to 18 carbon atoms. In one embodiment where y is greater than 1, the R₁ substituent on each chain is equivalent. Suitable examples of R₁ include methyl or ethyl, and most preferably methyl.

The choice of end group may modify the performance of the oligolactic acid derivatives with respect to that of oligolactic acid. For physical and chemical reasons it may be preferable to modify the end group to affect stability, drug solubility, water affinity, interaction with the drug, etc. Such parameters may influence drug delivery rates. Preferred oligolactic acid derivatives as described herein contain at least one chain capped with an organocarbonyl group, and more preferably, with an acetyl group. Acylation can significantly enhance stability and reduce the hydrophilicity and water solubility of the biocompatible polymers when such properties are desired.

Each chain or chains in the oligolactic acid derivative is capped or bridged on one end by a headgroup, X. Suitable examples of the headgroup X are selected from the group consisting of —OR₁, —SR₁, —N(R₁)₂, and divalent or trivalent headgroups terminated in —O—, —N—, or —S.

A chain may be capped by a monovalent, divalent, or polyvalent organic moiety (each valence of the capping group being independently bonded to a chain) that does not contain hydrogen atoms capable of hydrogen bonding. The chain may also be capped at one end or both ends by a monovalent, divalent, or polyvalent group, selected from either an ionic group or a group that does contain hydrogen atoms capable of hydrogen bonding. Where y is equal to I the chain is typically capped by a monovalent headgroup. Suitable examples of monovalent groups that may cap or terminate the compound include —OH, —OR₁, —SR₁, —N(R₁)₂. Capping groups need not necessarily terminate the compound; rather, they can bridge chains, which is the case when y is greater than 1. Where y is greater than 1, two or more chains are bridged by a divalent or polyvalent headgroup. For example, ethylenediamine is a suitable divalent headgroup capable of bridging two chains. In one embodiment, y is greater than or equal to 1 and less than or equal to 3. In one embodiment, y is 2.

Examples of groups not containing hydrogen atoms capable of hydrogen bonding include organocarbonyl groups such as acetyl and alkoxy groups such as ethoxy. Examples of ionic groups include quaternary ammonium groups, sulfonate salts, carboxylate salts, and the like. Examples of groups capable of hydrogen bonding include hydrogen when bonded to a heteroatom terminus of a chain, as well as acid functional groups, amides, carbamates, and groups such as amino, hydroxyl, thiol, aminoalkyl, alkylamino, hydroxyalkyl, hydroxyalkylamino, sugar residues, and the like. Such end groups are well known and can be readily selected by those skilled in the art, and are disclosed, for example, in U.S. Pat. Nos. 5,569,450 and 6,042,811, the disclosures of which are herein incorporated by reference.

In one embodiment, the number of repeat units, n, is greater than or equal to 3. In one embodiment, the number of repeat units, n, is less than or equal to 20, often less than or equal to 10, and sometimes less than or equal to 6. In one embodiment, the number of repeat units, n, is between about 3 and 20, often between about 3 and 10, and sometimes between about 3 and 6.

In one embodiment, the number-average number of repeat units (readily determinable by NMR analysis) is greater than or equal to 3. In one embodiment, the number-average number of repeat units is less than or equal to 20, often less than or equal to 10, and sometimes less than or equal to 6. In one embodiment, the number-average number of repeat units is between about 3 and 20, often between about 3 and 10, and sometimes between about 3 and 6.

In one embodiment, the number-average molecular weight (readily determinable by GPC analysis) is greater than about 150 g/mol, often greater than about 200 g/mol. In one embodiment, the number-average molecular weight (readily determinable by GPC analysis) is less than about 1500 g/mol, often less than about 1000 g/mol, and sometimes less than about 500 g/mol. In one embodiment, the number-average molecular weight is between about 150 and 1500 g/mol, often between about 200 and 1000 g/mol, and sometimes between about 200 and 500 g/mol.

A chain can be formally derived from any combination of L-lactic acid and D-lactic acid units. The chains can possess any sequence of units that can be derived from L-lactic acid and D-lactic acid. The sequence of the units derived from L- and D-isomers can be random or can have a contiguous sequence of a single isomer for a portion or entire chain length. The sequence may also possess a repeating structure comprising units derived from both L- and D-lactic acid units. In the embodiment where y is greater than 1, each sequence of units in the biocompatible compound may have a different isomeric composition.

In one embodiment, the precursor hydroxy-acid may be a mixture of L-lactic acid and D-lactic acid. In one embodiment, the precursor hydroxyacid is DL-lactic acid. The chain of units may contain any ratio of units derived from D-lactic acid and L-lactic acid, for example, ranging from 10:1; 1:1, or 1:10. The preferred ratio of units derived from D-lactic acid and L-lactic acid in the chain of units of formulas I and II is 1:1. The DL form may be advantageous due to its amorphous nature. The L form may also be advantageous as it is endogenous to the human body.

The amount of oligomeric adjuvant is typically greater than about 5% and sometimes greater than about 10%, by weight of the total weight of adhesive, drospirenone, and oligomeric adjuvant in the device. The amount of oligomeric adjuvant is typically less than about 30% and sometimes less than about 20%, by weight of the total weight of adhesive, drospirenone, and oligomeric adjuvant in the device. In one embodiment, the amount of oligomeric adjuvant is between about 5 and about 30%, sometimes between about 10 and about 20%, by weight of the total weight of adhesive, drospirenone, and oligomeric adjuvant in the device.

In one embodiment, the oligomeric adjuvant is a permeation enhancing adjuvant. A permeation enhancing adjuvant is an adjuvant which increases the amount of drug delivered transdermally for some portion of the delivery period of the device when compared to a like device without the adjuvant. For example, the permeation enhancing adjuvant may make the skin more permeable to transport of the drug and thus increase the rate at which the drug can pass through the skin. In another example, the permeation enhancing adjuvant may alter the affinity of the drug for the device, thereby increasing the thermodynamic potential driving the drug from the device into and through the skin.

The oligomeric adjuvant may be prepared according to any conventional synthetic method. Examples of suitable synthetic methods for preparing the oligomeric adjuvant may be found in U.S. patent application Nos. 60/533,172 (“Medicinal Compositions and Method for the Preparation Thereof”, Capecchi et al.) and 60/613,063 (“Medicinal Aerosol Formulations and Methods of Synthesizing Ingredients Therefor”, Bechtold et al.), the disclosures of which are herein incorporated by reference.

In one embodiment, transdermal drug delivery devices of the present invention comprise a backing film. Typical examples of flexible films employed as conventional tape backings which may be useful as a backing film include those made from polymer films such as polypropylene; polyethylene, particularly low density polyethylene, linear low density polyethylene, metallocene polyethylenes, and high density polyethylene; polyvinyl chloride; polyester (e.g., polyethylene terephthalate); polyvinylidene chloride; ethylene-vinyl acetate (EVA) copolymer; polyurethane; cellulose acetate; and ethyl cellulose. Coextruded multilayer polymeric films are also suitable, such as those described in U.S. Pat. No. 5,783,269 (Heilmann et al.), the disclosure of which is incorporated herein by reference. Backings that are layered such as polyethylene terephthalate-aluminum-polyethylene composites and polyethylene terephthalate-EVA composites are also suitable. Foam tape backings, such as closed cell polyolefin films used in 3M™ 1777 Foam Tape and 3M™ 1779 Foam Tape are also suitable. Polyethylenes, polyethylene blends, and polypropylenes are preferred polymer films. Polyethylenes and polyethylene blends are most preferred polymer films. In one embodiment, the backing film is translucent or transparent. Additives may also be added to the backing film, such as tackifiers, plasticizers, colorants, ultraviolet absorbers, and anti-oxidants. It may be desirable to use a flexible backing film, particularly for medical or pharmaceutical applications where the end use product is adhered to skin. In one embodiment, the present method finds particular utility for island placement converting of adhesive laminates having very flexible backings, such as thin polyethylene backings, which are generally difficult to handle in small, individual patch shaped sections.

In one embodiment, the backing film thickness is more than 10 μm, often more than 20 μm, and sometimes more than 40 μm. In another embodiment, the backing film thickness is less than 2 mm, often less than 1 mm, and sometimes less than 150 μm.

Transdermal drug delivery devices of the present invention may take any number of conventional forms. Suitable transdermal drug delivery devices include gelled or liquid reservoirs, such as in U.S. Pat. No. 4,834,979 (Gale), so-called “reservoir” patches; devices containing matrix reservoirs attached to the skin by an adjacent adhesive layer, such as in U.S. Pat. No. 6,004,578 (Lee, et al.), so-called “matrix” patches; and devices containing pressure-sensitive adhesive reservoirs, such as in U.S. Pat. Nos. 6,365,178 (Venkateshwaran et al.), 6,024,976 (Miranda et al.), and 6,149,935 (Chiang et al.), so-called “drug-in-adhesive” patches, the disclosures of which are incorporated herein by reference.

Transdermal drug delivery devices of the present invention comprise an effective amount of drospirenone. This amount will vary according to the form of the drug used, the particular condition to be treated, the amount of time the composition is allowed to remain in contact with the skin of the subject, the efficiency with which drug is delivered from the device to a patient, and other factors known to those of skill in the art. Devices will typically contain from about 1 mg to about 50 mg of drospirenone. In one embodiment, devices will contain more than about 10 mg of drospirenone and sometimes more than about 20 mg. An effective amount for contraception will typically be sufficient to deliver between about 1 and 3 mg/day to a patient. In one embodiment the device will contain a sufficient amount of drospirenone to deliver an effective amount for a period of at least 7 days. In a typical device of the present invention the amount of drug in the device will be about 1.2 to 4 times the amount of drug delivered to the patient. The amount of drospirenone is typically between about 5 and about 15%, sometimes between about 10 and about 15%, by weight of the total weight of adhesive, drospirenone, and oligomeric adjuvant in the device.

In one embodiment the rate (or flux) of drug delivered transdermally will be relatively constant during the wear period, (i.e., the time that the composition remains in contact with the skin) exclusive of the lag time as discussed below. It should be understood that typical transdermal devices have a lag time, that is, an initial time during the wear period before the transdermal delivery reaches peak flux. This lag time is typically between 1 and 24 hours for most transdermal delivery devices, and as such, the drug flux during the lag time may not be relatively constant. In this embodiment, the rate of drug delivered during the remainder of the wear period (i.e., after reaching peak flux) will be relatively constant. In particular, the rate of drug delivered at the end of the wear period will not be significantly less than the peak flux achieved during the wear period. In one embodiment the transdermal flux of drospirenone at the end of the wear period is greater than or equal to 50% of the peak flux of drospirenone during the wear period of the device. In one embodiment the transdermal flux of a second drug, such as an estrogenic compound, at the end of the wear period is greater than or equal to 50% of the peak flux of the second drug during the wear period of the device.

The devices are preferably “drug-in-adhesive” type devices and as such, the drospirenone and any other drugs are dispersed within the skin contacting adhesive. Drospirenone and any other drugs may be dispersed within any portion of the skin contacting adhesive and may be present in dissolved and/or undissolved (i.e., particulate) form. In one embodiment the drospirenone is homogeneously dispersed within the skin contacting adhesive. For example, drospirenone may be present as small particles that are evenly (or homogeneously) mixed throughout the adhesive or the drospirenone may be dissolved within the adhesive so that the concentration of dissolved drospirenone is constant throughout the adhesive. The drospirenone is preferably dissolved within the skin contacting adhesive and in one embodiment, the adhesive matrix is substantially free of undissolved drospirenone, and preferably free of undissolved drospirenone. The presence of undissolved drospirenone may be detected, for example, by examination with an optical microscope at 20× magnification. It should be understood that where only an occasional crystal or undissolved particle is present or where less than about 1% of the total amount of drospirenone is undissolved, the composition is considered to be substantially free of undissolved drospirenone.

In one aspect, devices of the present invention comprise a solubilizing agent. A solubilizing agent is a generally low molecular weight compound (i.e., typically with a molecular weight of 2000 g/mol or less) that may be added to an adhesive in order to increase the solubility of a drug within the device. The solubility of drug, such as drospirenone, will be greater in the solubilizing agent than it is in the adhesive, and thus a mixture of adhesive and solubilizing agent will be able to dissolve a higher concentration of drug than the adhesive can alone. The solubility of drospirenone in the solubilizing agent is typically greater than 5% by weight, often greater than 10% by weight, and sometimes greater than 20% by weight. Solubility of drospirenone may be determined by any of a number of conventional methods. For example, solubility in a liquid solubilizing agent may be determined by adding an excess of drug to the liquid and mixing to allow the maximum amount of drug to dissolve into the liquid. The excess, undissolved drug is then filtered from the solution and the solution is analyzed for dissolved drug concentration. Solubility in an adhesive matrix may be determined, for example, by preparing sheets of adhesive with varying concentrations of drug and observing them over time to see if drug crystallizes. The solubility is then considered to be the highest concentration of drug that can be added to the sheet without leading to crystallization on extended storage (e.g., 3-, 6-, 12-, or 24-month storage).

The amount of solubilizing agent is typically between about 5 and about 30%, sometimes between about 10 and about 20%, by weight of the total weight of adhesive, drospirenone, and solubilizing agent in the device.

In one aspect, devices of the present invention comprise an aromatic solubilizing agent. Aromatic as a term refers to ring structures containing only carbon, examples of which are phenyl or naphthyl groups. Examples of suitable aromatic solubilizing agents for drospirenone include benzyl alcohol, benzyl lactate, benzyl benzoate, 1-phenyl-1-propanol, 2-phenyl-2-propanol, and methyl salicylate.

The amount of aromatic solubilizing agent is typically between about 5 and about 30%, sometimes between about 10 and about 20%, by weight of the total weight of adhesive, drospirenone, and aromatic solubilizing agent in the device.

In one aspect, devices of the present invention comprise a permeation enhancer other than oligolactic acid or oligolactic acid derivatives. Examples of suitable enhancers include terpenes, such as α-terpineol, terpinolene, α-terpinene, terpinene-4-ol, and cineole; lactates, such as lauryl lactate, methyl lactate, and benzyl lactate; carboxylic acids, such as hydroxyacids (e.g., lactic acid, citric acid, and mandelic acid) and benzoic acid; dimethyl sulfoxide; alkyl esters of fatty acids, such as methyl laurate, isopropyl myristate, and ethyl oleate; monoglycerides of C₈-C₃₆ fatty acids such as glyceryl monooleate and glyceryl monolaurate; tetraglycol (tetrahydrofurfuryl alcohol polyethylene glycol ether); dipropylene glycol; triacetin, 2-(2-ethoxyethoxy)ethanol; and combinations of the foregoing. Alkyl lactates, carboxylic acids, and methyl laurate are preferred permeation enhancers. In one embodiment, the carboxylic acid is a hydroxy acid. Alpha-hydroxy acids (i.e., acids with the formula HO—C(R₂)(R₃)—COOH) are preferred hydroxyacids. It may be desirable to combine a permeation enhancer with an aromatic solubilizing agent in an adhesive matrix of the present invention. A single permeation enhancer may be used or a combination of more than one may be used. Other enhancers that may also be suitable include those disclosed in U.S. Pat. No. 6,024,976, the disclosure of which is hereby incorporated by reference. Examples of other optional additives include tackifiers, plasticizers, and anti-oxidants.

The amount of permeation enhancer is typically between about 1 and about 30%, sometimes between about 4 and about 20%, by weight of the total weight of adhesive, drospirenone, and aromatic solubilizing agent in the device. In one embodiment, the amount of carboxylic acid permeation enhancer is between about 1 and about 10%, sometimes between about 4 and 8%, by weight of the total weight of adhesive, drospirenone, and aromatic solubilizing agent in the device.

In one embodiment, a carboxylic acid permeation enhancer may be combined with an alcohol solubilizing agent, such as benzyl alcohol. Under some conditions, such a combination may lead to an esterification of the carboxylic acid. In one embodiment it may be desirable to prepare an initial formulation having an amount of the carboxylic acid ester present, so as to inhibit or minimize the effect of any carboxylic acid ester that may be formed during storage. In one embodiment, enhancer and solubilizing agent may be selected such that substantially no ester (e.g., benzyl benzoate) is formed. For example, a formulation combining benzyl alcohol and benzoic acid may, under certain conditions, form benzyl benzoate which in itself may be desirable to have in the formulation as a solubilizing agent.

The length of time that the device remains in a delivering relationship is typically an extended time, for example, from about 12 hours to about 14 days. In certain embodiments, the length of time that the reservoir remains in a delivering relationship is about 1 day (i.e., daily dosing), about 3 to 4 days (bi-weekly dosing), or about 7 days (weekly dosing).

Devices of the present invention may further comprise another drug, such as an estrogenic compound to be delivered in combination with drospirenone. Examples of estrogens which can be employed in this invention are ethinyl estradiol, mestranol, estradiol and its esters, e.g., valerate, acetate, benzoate and undecylate, estriol, estriol succinate, polyestriol phosphate, estrone, estrone sulfate and conjugated estrogens. Ethinyl estradiol is preferred. The amount of estrogen in the device will vary according to the type, the particular condition to be treated, the amount of time the composition is allowed to remain in contact with the skin of the subject, and other factors known to those of skill in the art. The amount and concentration of ethinyl estradiol will typically be much less than that of drospirenone, with typical total amounts in a device being between about 50 μg and 500 μg.

Generally, the device will be in the form of a patch with a size suitable to deliver a selected amount of drug through the skin. The device will typically have a surface area greater than about 1 cm², and sometimes greater than about 5 cm². Generally, the device will have a surface area of less than about 100 cm², often less than about 40 cm², and sometimes less than about 25 cm². In one embodiment, devices may be packaged individually in a foil-lined pouch for storage. In one embodiment, devices may alternatively be provided in a rolled or stacked form suitable for use with a dispensing apparatus.

Dosage forms of the present invention typically comprise a release liner that covers and protects the skin-contacting surface prior to use by a patient. Suitable release liners include conventional release liners comprising a known sheet material such as a polyester web, a polyethylene web, a polypropylene web, or a polyethylene-coated paper coated with a suitable fluoropolymer or silicone based coating. In one embodiment the release liner is the same shape and size as the area of the adhesive portion of the device. It may be desirable to have one or more cuts or splits in the release liner to assist in removal of the adhesive portion from the liner. In one embodiment the release liner has a larger area than the adhesive portion of the device, thereby providing an extended liner. The distance that the release liner extends beyond the margins of the adhesive portion of the device can be any suitable distance, and may depend upon a number of factors including, for example, the size of the adhesive portion of the patch, the types of adhesive, backing, and liner employed, and the patient population using the patch. The distance that the liner extends may be uniform around the circumference of the patch or it may vary, for example, by providing a smaller circular patch on a square-shaped extended liner.

In one embodiment, transdermal drug delivery devices of the invention are prepared by combining the copolymer, drug, and any adjuvants or permeation enhancers with an organic solvent (e.g., ethyl acetate, isopropanol, methanol, acetone, 2-butanone, ethanol, toluene, alkanes, and mixtures thereof) to provide a coating composition. The mixture is shaken or stirred until a homogeneous coating composition is obtained. The resulting composition is then applied to a release liner using conventional coating methods (e.g., knife coating or extrusion die coating) to provide a predetermined uniform thickness of coating composition. The release liner that has been coated with the composition is then dried and laminated onto a backing using conventional methods.

Devices of the present invention may be used for providing contraception to a human female. The device may be placed in a delivering relationship to the skin of a human female for an appropriate time period. In one embodiment the device has a total surface area of no more than 25 cm² and comprises a pressure sensitive adhesive matrix having at least about 20 mg of dissolved drospirenone. The device is allowed to remain in place and delivers about 1 to 3 mg/day for an extended time, for example, from about 12 hours to about 14 days. In certain embodiments, the length of time that the reservoir remains in a delivering relationship is about 1 day (i.e., daily dosing), about 3 to 4 days (bi-weekly dosing), or about 7 days (weekly dosing). The device may further comprise ethinyl estradiol which is delivered in an amount of about 0.01 to 0.03 mg/day. It should be understood that the amounts delivered are average daily amounts over the total delivery period and that the actual amount delivered during any single given 24 hour time period may vary somewhat with respect to these values. The following examples are not intended to be limiting in scope of the present invention, but merely representative of devices suitable for providing drospirenone and/or ethinyl estradiol serum levels effective for providing contraception.

### EXAMPLES

### In Vitro Skin Permeation Test Method

The skin permeation data given in the examples below was obtained using the following test method. A 1.0 cm² transdermal patch was die-cut from a larger sheet for use as the test sample. The release liner was removed, and the patch was applied to the stratum corneum surface of human cadaver skin and pressed to cause uniform contact with the skin. The resulting patch/skin laminate was placed patch side up across the orifice of the lower portion of a vertical diffusion cell. The diffusion cell was assembled and the lower portion filled with 5 mL of warm (32° C.) receptor fluid (30% (v/v) N-methyl-2-pyrrolidone in water) so that the receptor fluid contacted the skin. The sampling port was covered except when in use.

The cells were maintained at 32±1° C. throughout the course of the experiment. The receptor fluid was stirred by means of a magnetic stirrer throughout the experiment to assure a uniform sample and a reduced diffusion barrier on the dermal side of the skin. The entire volume of receptor fluid was withdrawn at specified time intervals and immediately replaced with fresh fluid. The withdrawn fluid was analyzed for drug using conventional high performance liquid chromatography methods. The cumulative (or total) flux of drug penetrating through the skin was calculated and reported as μg/cm². Unless noted, the results are reported as the average of 4 to 6 replicates. Unless noted, the total flux was determined after a time period of 7 days.

### Drospirenone Solubility Test Method

The solubility of drospirenone in liquid excipients was obtained using the following test method. Drospirenone was incrementally added to excipient until it stopped dissolving. The saturated solution was then filtered through a 0.45 μm polytetrafluoroethylene filter to remove excess drug particles. The concentration of drospirenone was then analyzed for drug using conventional high performance liquid chromatography methods. The values are reported as the percent solubility on a weight-weight basis.

The solubility of drospirenone in glyceryl monooleate was 2.0%. The solubility of drospirenone in methyl laurate was 0.6%. The solubility of drospirenone in benzyl alcohol was 44.4%. The solubility of drospirenone in methyl lactate was 11.6%. The solubility of drospirenone in lauryl lactate was 1.4%. The solubility of drospirenone in benzyl lactate was 22.6%. The solubility of drospirenone in lauryl alcohol was 1.0%.

### Probe Tack Test Method

The tack data given in the examples below was obtained using a Digital Polyken Probe Tack Tester, Model 80-02-01 (Testing Machines, Inc., Amityville, N.Y.). The machine settings were as follows: separation rate: 0.5 cm/second, contact time: 2 seconds; contact pressure: 100 g/cm². A stainless steel probe was used. The result of the test is the force required to break the bond between the probe and the surface of the test sample. The force is measured in “grams of tack”.

### Shear Creep Compliance Test Method

The compliance values given in the examples below were obtained using a modified version of the Creep Compliance Procedure described in U.S. Pat. No. 4,737,559 (Kellen). The release liner is removed from a sample of the material to be tested. The exposed surface is folded back on itself in the lengthwise direction to produce a “sandwich” configuration (i.e., backing/pressure sensitive adhesive/backing). The sandwich sample is passed through a laminator. Two test samples of equal area are cut from the laminated sandwich using a die. One test sample is centered on the stationary plate of a shear-creep rheometer. The small, non-stationary plate of the shear-creep rheometer is centered over the first sample on the stationary plate such that the hook is facing up and toward the front of the rheometer. The second test sample is centered on the upper surface of the small, non-stationary plate. The large non-stationary plate is placed over the second test sample and the entire assembly is clamped into place. A string is connected to the hook of the small, non-stationary plate and extended over the front pulley of the rheometer. A weight (e.g., 500 g) is attached to the free end of the string and supported so as not to place a load on the non-stationary plate. The support for the weight is removed to allow it to hang freely. The weight exerts a load on the non-stationary plate and the displacement of the non-stationary plate is recorded as a function of time. The weight is removed after exactly 3 minutes have elapsed. The shear creep compliance is then calculated using the equation:

$$J = 2 \frac{A X}{h f}$$

where A is the area of one face of the test sample, h is the thickness of the pressure sensitive adhesive mass (i.e., two times the thickness of the pressure sensitive adhesive layer on each sandwich), X is the displacement and f is the force due to the mass attached to the string. Where A is expressed in cm², h in cm, X in cm and f in dynes, the compliance value is given in cm²/dyne.

### Preparation of “Dried” Copolymer

Dried copolymer was prepared by knife coating a solution of the copolymer onto a release liner. The coated release liner was oven dried to remove the solvent and reduce the level of residual monomers. The dried copolymer was then stripped off of the release liner and stored in a container until used.

### Copolymer D1

### Preparation of Isooctyl Acrylate/Acrylamide/Vinyl Acetate (75/5/20) Copolymer

A solution of isooctyl acrylate, acrylamide, and vinyl acetate (75:5:20) copolymer was prepared according to the general method described in Part D of U.S. Pat. No. 5,223,261, the disclosure of which is hereby incorporated by reference. The resulting copolymer solution was approximately 32% solids in a 90:10 blend of ethyl acetate and methanol. The inherent viscosity (IV) of the copolymer solution was approximately 1.35 dL/g.

### Copolymer D2

### Preparation of Isooctyl Acrylate/Acrylamide/Vinyl Acetate (71/9/20) Copolymer

A solution of isooctyl acrylate, acrylamide, and vinyl acetate (71:9:20) copolymer was prepared according to the general method described in Example 1 of U.S. Patent Application Publication No. 2003-054025, the disclosure of which is hereby incorporated by reference. The resulting copolymer solution was 16.4% solids in an 80:20 blend of ethyl acetate and methanol. The inherent viscosity (IV) of the copolymer solution was 1.53 dL/g.

### Copolymer D3

### Preparation of Isooctyl Acrylate/Acrylamide/Vinyl Acetate (70/10/20) Copolymer

A solution was prepared by combining isooctyl acrylate (140 g), acrylamide (20 g), vinyl acetate (4 g), 2,2′-azobis(2-methylbutyronitrile) (0.3 g), ethyl acetate (203.6 g) and methanol (58.9 g) in a 1 quart (0.95 L) amber glass bottle. The bottle was purged for 2 minutes with nitrogen at a flow rate of 1 L per minute. The bottle was sealed and placed in a rotating water bath at 57° C. for 24 hours. The resulting copolymer was diluted with ethyl acetate (169.7 g) and methanol (49.4 g) to 30% solids. The inherent viscosity was 1.17 dL/g.

### Example 1

Drospirenone (0.3933 g), methyl laurate (0.3655 g), and glyceryl monooleate (0.6083 g) were added to a solvent mixture of ethyl acetate (5.9515 g) and acetone (1.8472 g) and mixed until the drospirenone was dissolved. To this solution, copolymer (2.0746 g of dried Isooctyl acrylate/Acrylamide/Vinyl Acetate (75/5/20) from Copolymer D1 above) and oligolactic acid of formula I with an average value of n of 3.7 (0.5851 g) were added and mixed for at least 12 hours and until a uniform coating formulation was obtained. The coating formulation was knife coated at a wet thickness of 25 mil (635 μm) onto a silicone coated release liner. The coated liner was oven dried for 10 minutes at 110° F. (43° C.). The dried formulation was then laminated onto a backing (SCOTCHPAK™ 9732 polyester film laminate; available from 3M Company). The resulting formulation had a nominal composition of 9.1% methyl laurate, 15.1% glyceryl monooleate, 9.8% drospirenone, and 14.5% oligolactic acid with n=3.7. The remainder of the formulation was copolymer D1. The dry coating weight was approximately 16.9 mg/cm². The permeation through human cadaver skin was determined using the test method described above. The total drospirenone flux was 500±30 μg/cm.

### Example 2

Drospirenone (0.4085 g) and methyl laurate (0.9845 g) were added to a solvent mixture of ethyl acetate (6.2665 g) and acetone (1.8660 g) and mixed until the drospirenone was dissolved. To this solution, copolymer (2.0859 g of dried Isooctyl acrylate/Acrylamide/Vinyl Acetate (75/5/20) from Copolymer D1 above) and oligolactic acid of formula I with an average value of n of 3.7 (0.5608 g) were added and mixed for at least 12 hours and until a uniform coating formulation was obtained. The coating formulation was knife coated at a wet thickness of 25 mil (635 μm) onto a silicone coated release liner. The coated liner was oven dried for 10 minutes at 110° F. (43° C.). The dried formulation was then laminated onto a backing (SCOTCHPAK™ 9732 polyester film laminate; available from 3M Company). The resulting formulation had a nominal composition of 24.4% methyl laurate, 10.1% drospirenone, and 13.9% oligolactic acid with n=3.7. The remainder of the formulation was copolymer DI. The dry coating weight was 17.6 mg/cm². The permeation through human cadaver skin was determined using the test method described above. The total drospirenone flux was 470±130 μg/cm².

### Example 3

Drospirenone (0.284 g), methyl laurate (0.337 g), and benzyl alcohol (0.417 g) were added to a 70/30 (w/w) ethyl acetate/methanol solvent mixture (7.2 g) and mixed until the drospirenone was dissolved. To this solution, copolymer (1.648 g of dried Isooctyl acrylate/Acrylamide/Vinyl Acetate (70/10/20) from Copolymer D3 above), oligolactic acid of formula I with an average value of n of 3.7 (0.512 g), and oligolactic acid of formula I with an average value of n of 5.2 (0.213 g), were added and mixed for at least 12 hours and until a uniform coating formulation was obtained. The coating formulation was knife coated at a wet thickness of 25 mil (635 μm) onto a silicone coated release liner. The coated liner was oven dried for 10 minutes at 110° F. (43° C.). The dried formulation was then laminated onto a backing (SCOTCHPAK™ 9732 polyester film laminate; available from 3M Company). The resulting formulation had a nominal composition of 9.9% methyl laurate, 12.2% benzyl alcohol, 8.3% drospirenone, 15.0% oligolactic acid with n=3.7, and 6.2% oligolactic acid with n=5.2. The remainder of the formulation was copolymer D3. The dry coating weight was approximately 15 to 20 mg/cm². The permeation through human cadaver skin was determined using the test method described above. The total drospirenone flux was 670±250 μg/cm².

### Example 4

Drospirenone (0.282 g), methyl lactate (0.343 g), and benzyl alcohol (0.454 g) were added to a 70/30 (w/w) ethyl acetate/methanol solvent mixture (5.66 g) and mixed until the drospirenone was dissolved. To this solution, copolymer (1.588 g of dried Isooctyl acrylate/Acrylamide/Vinyl Acetate (70/10/20) from Copolymer D3 above), oligolactic acid of formula I with an average value of n of 3.7 (0.384 g), and oligolactic acid of formula I with an average value of n of 5.2 (0.354 g), were added and mixed for at least 12 hours and until a uniform coating formulation was obtained. The coating formulation was knife coated at a wet thickness of 25 mil (635 μm) onto a silicone coated release liner. The coated liner was oven dried for 10 minutes at 110° F. (43° C.). The dried formulation was then laminated onto a backing (SCOTCHPAK™ 9732 polyester film laminate; available from 3M Company). The resulting formulation had a nominal composition of 10.1% methyl lactate, 13.3% benzyl alcohol, 8.3% drospirenone, 11.3% oligolactic acid with n=3.7, and 10.4% oligolactic acid with n=5.2. The remainder of the formulation was copolymer D3. The dry coating weight was approximately 15 to 20 mg/cm². The total drospirenone flux was 360±130 μg/cm².

### Example 5

Drospirenone (0.2974 g), ethinyl estradiol (0.0178 g), and glyceryl monooleate (0.8088 g) were added to a 70/30 (w/w) ethyl acetate/methanol solvent mixture (4.81 g) and mixed until the drospirenone and ethinyl estradiol was dissolved. To this solution, copolymer (1.34 g of dried Isooctyl acrylate/Acrylamide/Vinyl Acetate (71/9/20) from Copolymer D2 above), oligolactic acid of formula I with an average value of n of 3.7 (0.37 g), oligolactic acid of formula I with an average value of n of 5.2 (0.11 g), and oligolactic acid of formula I with an average value of n of 10.6 (0.13 g) were added and mixed until a uniform coating formulation was obtained. The coating formulation was knife coated at a wet thickness of 25 mil (635 m) onto a silicone coated release liner. The coated liner was oven dried for 10 minutes at 110° F. (43° C.). The dried formulation was then laminated onto a backing (SCOTCHPAK™ 9732 polyester film laminate; available from 3M Company). The resulting formulation had a nominal composition of 25.3% glyceryl monooleate, 12.0% oligolactic acid with n=3.7, 3.6% oligolactic acid with n=5.2, and 4.2% oligolactic acid with n=10.6. The concentration of drospirenone and ethinyl estradiol were analytically determined by HPLC to be 7.0% and 0.39%, respectively, by weight of the total composition. The remainder of the formulation was copolymer D3. The dry coating weight was approximately 20 mg/cm². The permeation through human cadaver skin was determined using the test method described above. The total drospirenone flux and ethinyl estradiol flux is reported in Table 1.

### Examples 6-35

Samples were prepared according to the procedure of Example 5 with the exception that the composition of the liquid excipient was varied. The amount of drospirenone and ethinyl estradiol were adjusted to account for their relative solubility in the liquid excipient mixtures. The formulation compositions are shown in Table 1. All formulations had a nominal composition of 12.0% oligolactic acid with n=3.7, 3.6% oligolactic acid with n=5.2, and 4.2% oligolactic acid with n=10.6. Except where noted, the amounts of drospirenone and ethinyl estradiol were analytically determined by high performance liquid chromatography and the amounts of liquid excipient were analytically determined by gas chromatography. The permeation through human cadaver skin was determined using the test method described above. The total drospirenone flux and ethinyl estradiol flux is reported in Table 1. Cumulative drospirenone flux as a function of time is reported in Table 2. Probe tack and shear creep compliance were measured on selected samples and the results are reported in Table 3.

TABLE 1

| | | | | | | | | | | Ethinyl |
|---|---|---|---|---|---|---|---|---|---|---|
| | Glyceryl | Lauryl | Benzyl | Methyl | Benzyl | Methyl | | Ethinyl | Drospirenone | estradiol |
| Ex. | Monooleate | lactate | alcohol | S-lactate | lactate | Laurate | Drospirenone | estradiol | Total Flux | Total Flux |
| No. | [% w/w]* | [% w/w] | [% w/w] | [% w/w] | [% w/w] | [% w/w] | [% w/w] | [% w/w] | [μg/cm²] | [μg/cm²] |
| 5 | 25.3 | — | — | — | — | — | 7.0 | 0.39 | 820 ± 180 | 41 ± 8 |
| 6 | 12.8 | 16.3 | — | — | — | — | 8.5 | 0.37 | 970 ± 140 | 44 ± 6 |
| 7 | 12.8 | — | 12.6 | — | — | — | 9.6 | 0.48 | 920 ± 70 | 41 ± 5 |
| 8 | — | 32.0 | — | — | — | — | 8.9 | 0.42 | 850 ± 330 | 47 ± 16 |
| 9 | — | 19.0 | 12.2 | — | — | — | 10.9 | 0.53 | 990 ± 340 | 49 ± 10 |
| 10 | — | — | 21.3 | — | — | — | 9.0 | 0.52 | 670 ± 200 | 37 ± 5 |
| 11 | 8.5 | 10.5 | 7.6 | — | — | — | 8.8 | 0.50 | 890 ± 200 | 52 ± 13 |
| 12 | 17.3 | 5.8 | 3.7 | — | — | — | 8.9 | 0.40 | 790 ± 110 | 39 ± 4 |
| 13 | 4.1 | 19.6 | 3.4 | — | — | — | 8.7 | 0.39 | 940 ± 220 | 44 ± 5 |
| 14 | 4.7 | 6.1 | 16.1 | — | — | — | 10.4 | 0.57 | 990 ± 300 | 53 ± 18 |
| 15 | — | — | — | 10.8 | — | — | 11.8 | 0.61 | 550 ± 310 | 31 ± 14 |
| 16 | — | 14.0 | — | 2.7 | — | — | 7.8 | 0.36 | 910 ± 170 | 51 ± 12 |
| 17 | — | — | 15.6 | 6.2 | — | — | 12.7 | 0.71 | 830 ± 130 | 41 ± 8 |
| 18 | — | 11.9 | 8.3 | 2.7 | — | — | 10.1 | 0.47 | 890 ± 200 | 43 ± 9 |
| 19 | — | 4.4 | 3.3 | 5.5 | — | — | 8.4 | 0.47 | 750 ± 280 | 44 ± 15 |
| 20 | — | 21.0 | 3.4 | 1.1 | — | — | 9.0 | 0.47 | 1040 ± 100 | 54 ± 12 |
| 21 | — | 6.8 | 17.3 | 1.2 | — | — | 11.0 | 0.59 | 1070 ± 110 | 46 ± 7 |
| 22 | — | — | 19.0 | 1.9 | — | — | 10.7 | 0.48 | 630 ± 270 | 29 ± 10 |
| 23 | — | — | 5.2 | 4.9 | — | — | 8.5 | 0.41 | 560 ± 210 | 42 ± 8 |
| 24 | — | — | — | — | 22.1 | — | 7.8 | 0.23 | 520 ± 160 | 32 ± 12 |
| 25 | — | 15.8 | — | — | 13.0 | — | 8.9 | 0.51 | 890 ± 130 | 57 ± 10 |
| 26 | — | — | 9.8 | — | 11.2 | — | 8.2 | 0.54 | 870 ± 90 | 45 ± 6 |
| 27 | — | 8.6 | 6.7 | — | 7.3 | — | 7.9 | 0.54 | 850 ± 140 | 71 ± 15 |
| 28 | — | — | 8.4 | — | — | 16.3 | 7.2 | 0.45 | 810 ± 290 | 60 ± 13 |
| 29 | 11.7 | — | — | — | — | 19.0 | 8.2 | 0.36 | 720 ± 240 | 44 ± 7 |
| 30 | 9.0 | — | 7.0 | — | — | 9.8 | 8.9 | 0.47 | 760 ± 190 | 44 ± 4 |
| 31 | 4.3 | — | 14.4 | — | — | 5.0 | 7.8 | 0.39 | 960 ± 200 | 44 ± 6 |
| 32 | 4.9 | — | 8.2 | — | — | 14.5 | 7.7 | 0.48 | 830 ± 230 | 63 ± 10 |
| 33 | 16.8 | — | 4.0 | — | — | 6.2 | 7.6 | 0.45 | 740 ± 50 | 47 ± 8 |
| 34 | 11.1 | — | 3.5 | — | — | 15.4 | 7.4 | 0.42 | 970 ± 270 | 63 ± 7 |
| 35 | — | — | — | — | — | — | 12.2* | 0.61* | 590 ± 140 | 37 ± 9 |

*Nominal amount based on input amounts to formulation.

TABLE 2
Human Cadaver Skin Permeation

| Example | Average Cumulative Amount Penetrated (μg/cm²) | | | | | | |
|---|---|---|---|---|---|---|---|
| Number | 24 hr | 48 hr | 72 hr | 96 hr | 120 hr | 144 hr | 168 hr |
| 5 | 150 | 330 | 500 | 630 | 720 | 780 | 820 |
| 6 | 300 | 570 | 760 | 870 | 920 | 950 | 970 |
| 7 | 170 | 350 | 530 | 670 | 780 | 860 | 920 |
| 8 | 270 | 480 | 640 | 720 | 770 | 810 | 850 |
| 9 | 280 | 550 | 750 | 870 | 930 | 970 | 990 |
| 10 | 70 | 160 | 270 | 380 | 490 | 590 | 670 |
| 11 | 170 | 370 | 560 | 700 | 790 | 850 | 890 |
| 12 | 120 | 280 | 450 | 570 | 670 | 740 | 790 |
| 13 | 250 | 510 | 710 | 830 | 890 | 920 | 940 |
| 14 | 180 | 390 | 600 | 760 | 870 | 940 | 990 |
| 15 | 70 | 150 | 240 | 330 | 410 | 480 | 550 |
| 16 | 280 | 510 | 700 | 800 | 860 | 900 | 910 |
| 17 | 120 | 250 | 390 | 520 | 640 | 740 | 830 |
| 18 | 180 | 360 | 540 | 670 | 770 | 840 | 880 |
| 19 | 110 | 250 | 390 | 520 | 620 | 690 | 750 |
| 20 | 320 | 600 | 800 | 910 | 970 | 1010 | 1040 |
| 21 | 160 | 360 | 560 | 740 | 880 | 980 | 1070 |
| 22 | 60 | 140 | 240 | 340 | 450 | 540 | 630 |
| 23 | 70 | 150 | 240 | 340 | 420 | 490 | 560 |
| 24 | 90 | 180 | 270 | 340 | 410 | 460 | 520 |
| 25 | 230 | 460 | 640 | 750 | 820 | 860 | 890 |
| 26 | 90 | 220 | 370 | 510 | 650 | 770 | 870 |
| 27 | 190 | 360 | 520 | 640 | 730 | 800 | 850 |
| 28 | 330 | 530 | 650 | 730 | 770 | 800 | 810 |
| 29 | 260 | 440 | 560 | 630 | 670 | 700 | 720 |
| 30 | 150 | 310 | 460 | 580 | 660 | 710 | 760 |
| 31 | 170 | 360 | 550 | 700 | 820 | 900 | 960 |
| 32 | 290 | 500 | 630 | 720 | 770 | 800 | 830 |
| 33 | 130 | 280 | 430 | 540 | 640 | 700 | 740 |
| 34 | 340 | 580 | 750 | 850 | 910 | 940 | 970 |
| 35 | 70 | 170 | 270 | 360 | 450 | 520 | 590 |

TABLE 3

| | | Compliance |
|---|---|---|
| Example Number | Probe Tack [grams] | [x 10⁵ cm²/dyne] |
| 13 | 217 | 2.6 |
| 15 | 427 | 4.1 |
| 36 | 1025 | 0.5 |

### Example 36

Drospirenone (0.2938 g) and ethinyl estradiol (0.0160 g) were added to a 70/30 (w/w) ethyl acetate/methanol solvent mixture (9.271 g) and mixed until the drospirenone and ethinyl estradiol was dissolved. To this solution, copolymer (1.981 g of dried Isooctyl acrylate/Acrylamide/Vinyl Acetate (71/9/20) from Copolymer D2 above) and oligolactic acid of formula I with an average value of n of 3.7 (0.636 g) were added and mixed for at least 12 hours and until a uniform coating formulation was obtained. The coating formulation was knife coated at a wet thickness of 25 mil (635 μm) onto a silicone coated release liner. The coated liner was oven dried for 10 minutes at 110° F. (43° C.). The dried formulation was then laminated onto a backing (SCOTCHPAK™ 9732 polyester film laminate; available from 3M Company). The resulting formulation had a nominal composition of 0.55% ethinyl estradiol, 10.0% drospirenone, and 21.7% oligolactic acid with n=3.7. The remainder of the formulation was copolymer D2. The dry coating weight was 20 mg/cm². The probe tack force was determined to be 1025 g according to the procedure described above. The permeation through human cadaver skin was determined using the test method described above. The total drospirenone flux was 420±250 μg/cm² and the total ethinyl estradiol flux was 23±7 μg/cm².

### Example 37

A sample was prepared as in example 36 with the exception that the oligolactic acid was acetylated. The resulting formulation had a nominal composition of 0.52% ethinyl estradiol, 9.8% drospirenone, and 21.1% acetylated oligolactic acid. The remainder of the formulation was copolymer D2. The permeation through human cadaver skin was determined using the test method described above. The total drospirenone flux was 350±80 μg/cm² and the total ethinyl estradiol flux was 27±4 μg/cm².

### Example 38

A sample was prepared as in example 36 with the exception that the terminal carboxyl group of the oligolactic acid was functionalized with a benzyl group and the average value of n was about 3. The resulting formulation had a nominal composition of 0.54% ethinyl estradiol, 10.0% drospirenone, and 22.9% functionalized oligolactic acid. The remainder of the formulation was copolymer D2. The permeation through human cadaver skin was determined using the test method described above. The total drospirenone flux was 190±70 μg/cm² and the total ethinyl estradiol flux was 17±4 μg/cm².

### Example 39

Drospirenone (0.3003 g), methyl laurate (0.479 g), and benzyl alcohol (0.400 g) were added to a 70/30 (w/w) ethyl acetate/methanol solvent mixture (6.546 g) and mixed until the drospirenone was dissolved. To this solution, copolymer (1.63 g of dried Isooctyl acrylate/Acrylamide/Vinyl Acetate (75/5/20) from Copolymer D1 above) and oligolactic acid of formula I with an average value of n of 3.7 (0.363 g) were added and mixed for at least 12 hours and until a uniform coating formulation was obtained. The coating formulation was knife coated at a wet thickness of 25 mil (635 μm) onto a silicone coated release liner. The coated liner was oven dried for 10 minutes at 110° F. (43° C.). The dried formulation was then laminated onto a backing (SCOTCHPAK™ 9732 polyester film laminate; available from 3M Company). The resulting formulation had a nominal composition of 15.1% methyl laurate, 12.6% benzyl alcohol, 9.5% drospirenone, and 11.4% oligolactic acid with n=3.7. The remainder of the formulation was copolymer D1. The dry coating weight was approximately 15 to 20 mg/cm². The permeation through human cadaver skin was determined using the test method described above. The total drospirenone flux was 560±50 μg/cm².

### Example 40

Drospirenone (0.9038 g), ethinyl estradiol (0.0354 g), benzyl alcohol (2.5050 g), lauryl alcohol (0.6075 g), and citric acid (1.5165 g) were added to a 70/30 (w/w) ethyl acetate/methanol solvent mixture (15.0652 g) and mixed until the drospirenone and ethinyl estradiol was dissolved. To this solution, copolymer (5.0531 g of dried Isooctyl acrylate/Acrylamide/Vinyl Acetate (71/9/20) from Copolymer D2 above) was added and mixed until a uniform coating formulation was obtained. The coating formulation was knife coated at a wet thickness of 25 mil (635 μm) onto a silicone coated release liner. The coated liner was oven dried for 10 minutes at 110° F. (43° C.). The dried formulation was then laminated onto a backing (SCOTCHPAK™ 9732 polyester film laminate; available from 3M Company). The resulting formulation had a nominal composition of 0.33% ethinyl estradiol, 8.5% drospirenone, 23.6% benzyl alcohol, 5.7% lauryl alcohol, and 14.3% citric acid. The remainder of the formulation was copolymer D2. The permeation through human cadaver skin was determined using the test method described above. The total drospirenone flux after a period of 94 hours was 810±140 μg/cm².

### Example 41

Drospirenone (1.0309 g), ethinyl estradiol (0.0355 g), benzyl alcohol (2.5064 g), methyl laurate (1.0664 g), lauryl alcohol (0.6026 g), and citric acid (0.5033 g) were added to a 70/30 (w/w) ethyl acetate/methanol solvent mixture (15.0234 g) and mixed until the drospirenone and ethinyl estradiol was dissolved. To this solution, copolymer (4.9877 g of dried Isooctyl acrylate/Acrylamide/Vinyl Acetate (71/9/20) from Copolymer D2 above) was added and mixed until a uniform coating formulation was obtained. The coating formulation was knife coated at a wet thickness of 25 mil (635 μm) onto a silicone coated release liner. The coated liner was oven dried for 10 minutes at 110° F. (43° C.). The dried formulation was then laminated onto a backing (SCOTCHPAK™ 9732 polyester film laminate; available from 3M Company). The resulting formulation had a nominal composition of 0.30% ethinyl estradiol, 9.6% drospirenone, 23.4% benzyl alcohol, 9.9% methyl laurate, 5.6% lauryl alcohol, and 4.7% citric acid. The remainder of the formulation was copolymer D2. The permeation through human cadaver skin was determined using the test method described above. The total drospirenone flux after a period of 94 hours was 840±100 μg/cm².

### Example 42

Drospirenone (0.9556 g), ethinyl estradiol (0.0359 g), benzyl alcohol (2.4949 g), methyl laurate (0.5318 g), lauryl alcohol (1.2009 g), and citric acid (0.5158 g) were added to a 70/30 (w/w) ethyl acetate/methanol solvent mixture (15.0030 g) and mixed until the drospirenone and ethinyl estradiol was dissolved. To this solution, copolymer (5.0033 g of dried Isooctyl acrylate/Acrylamide/Vinyl Acetate (71/9/20) from Copolymer D2 above) was added and mixed until a uniform coating formulation was obtained. The coating formulation was knife coated at a wet thickness of 25 mil (635 μm) onto a silicone coated release liner. The coated liner was oven dried for 10 minutes at 110° F. (43° C.). The dried formulation was then laminated onto a backing (SCOTCHPAK™ 9732 polyester film laminate; available from 3M Company). The resulting formulation had a nominal composition of 0.30% ethinyl estradiol, 8.9% drospirenone, 23.2% benzyl alcohol, 5.0% methyl laurate, 11.2% lauryl alcohol, and 4.8% citric acid. The remainder of the formulation was copolymer D2. The permeation through human cadaver skin was determined using the test method described above. The total drospirenone flux after a period of 94 hours was 910±210 μg/cm².

### Example 43

Drospirenone (0.520 g), ethinyl estradiol (0.0040 g), benzyl alcohol (1.000 g), methyl laurate (0.515 g), and benzoic acid (0.320 g) were added to a 70/30 (w/w) ethyl acetate/methanol solvent mixture (8.1212 g) and mixed until the drospirenone and ethinyl estradiol was dissolved. To this solution, copolymer (1.8760 g of dried Isooctyl acrylate/Acrylamide/Vinyl Acetate (75/5/20) from Copolymer D1 above) was added and mixed until a uniform coating formulation was obtained. The coating formulation was knife coated at a wet thickness of 22 mil (559 μm) onto a silicone coated release liner. The coated liner was oven dried for 10 minutes at 110° F. (43° C.). The dried formulation was then laminated onto a backing (SCOTCHPAK™ 9732 polyester film laminate; available from 3M Company). The resulting formulation had a nominal composition of 0.10% ethinyl estradiol, 13.0% drospirenone, 20.0% benzyl alcohol, 12.0% methyl laurate, and 8.0% benzoic acid. The remainder of the formulation was copolymer D1. The permeation through human cadaver skin was determined using the test method described above. The total drospirenone flux after a period of 96 hours was 209±184 μg/cm². The total ethinyl estradiol flux after a period of 96 hours was 0.55±0.26 μg/cm². Cumulative drospirenone flux as a function of time is reported in Table 4.

TABLE 4
Human Cadaver Skin Permeation

| Example | Average Cumulative Amount Penetrated (μg/cm²) | | | |
|---|---|---|---|---|
| Number | 24 hr | 48 hr | 72 hr | 96 hr |
| 43 | 107 | 129 | 170 | 209 |
| 44 | 71 | 112 | 148 | 183 |
| 45 | 29 | 83 | 99 | 110 |
| 46 | 63 | 126 | 168 | 209 |
| 47 | 27 | 131 | 171 | 209 |

### Example 44

A sample was prepared as in Example 43, with the exception that the nominal amounts of methyl laurate and benzoic acid were 5.0% and 15.0%, respectively. The permeation through human cadaver skin was determined using the test method described above. The total drospirenone flux after a period of 96 hours was 183±65 μg/cm². The total ethinyl estradiol flux after a period of 96 hours was 0.53±0.22 μg/cm². Cumulative drospirenone flux as a function of time is reported in Table 4.

### Example 45

A sample was prepared as in Example 43, with the exception that the nominal amounts of methyl laurate and benzoic acid were 19.0% and 1.0%, respectively. The permeation through human cadaver skin was determined using the test method described above. The total drospirenone flux after a period of 96 hours was 110±51 μg/cm². The total ethinyl estradiol flux after a period of 96 hours was 0.78±0.57 μg/cm². Cumulative drospirenone flux as a function of time is reported in Table 4.

### Example 46

A sample was prepared as in Example 43, with the exception that the nominal amounts of methyl laurate and benzoic acid were 8.5% and 11.5%, respectively. The permeation through human cadaver skin was determined using the test method described above. The total drospirenone flux after a period of 96 hours was 209±78 μg/cm². The total ethinyl estradiol flux after a period of 96 hours was 0.66±0.35 μg/cm². Cumulative drospirenone flux as a function of time is reported in Table 4.

### Example 47

A sample was prepared as in Example 43, with the exception that the nominal amounts of methyl laurate and benzoic acid were 15.5% and 4.5%, respectively. The permeation through human cadaver skin was determined using the test method described above. The total drospirenone flux after a period of 96 hours was 209±88 μg/cm². The total ethinyl estradiol flux after a period of 96 hours was 0.66±0.47 μg/cm². Cumulative drospirenone flux as a function of time is reported in Table 4.

The present invention has been described with reference to several embodiments thereof. The foregoing detailed description and examples have been provided for clarity of understanding only, and no unnecessary limitations are to be understood therefrom. It will be apparent to those skilled in the art that many changes can be made to the described embodiments without departing from the spirit and scope of the invention. Thus, the scope of the invention should not be limited to the exact details of the compositions and structures described herein, but rather by the language of the claims that follow. Where a numerical parameter is described as having a range between two values it should be understood that the range is inclusive of the two values that define the range.

1. A transdermal drug delivery device comprising: an adhesive matrix; an effective amount of drospirenone; and an oligomeric adjuvant selected from an oligolactic acid, oligolactic acid derivatives, or mixtures thereof.

2. A transdermal drug delivery device according to claim 1 wherein the oligomeric adjuvant is a permeation enhancing adjuvant.

3. A transdermal drug delivery device according to claim 1 and further comprising a solubilizing agent.

4. A transdermal drug delivery device according to claim 1 and further comprising an aromatic solubilizing agent.

5. A transdermal drug delivery device according to claim 1 and further comprising a permeation enhancer selected from the group consisting of alkyl lactates, hydroxyacids, alkyl esters of fatty acids, and mixtures thereof.

6. A transdermal drug delivery device according to claim 5 wherein the permeation enhancer comprises methyl laurate.

7. A transdermal drug delivery device according to claim 1 and further comprising a backing film.

8. A transdermal drug delivery device according to claim 1 wherein the oligomeric adjuvant comprises oligolactic acid.

9. A transdermal drug delivery device according to claim 1 wherein the amount of oligomeric adjuvant is between about 5 and 30% by weight of the total weight of adhesive, drospirenone, and oligomeric adjuvant.

10. A transdermal drug delivery device according to claim 1 wherein the amount of drospirenone is between about 5 and 15% by weight of the total amount of adhesive, drospirenone, and oligomeric adjuvant.

11. A transdermal drug delivery device according to claim 1 wherein drospirenone is dispersed within the adhesive matrix.

12. A transdermal drug delivery device comprising: a backing film; and an adhesive matrix comprising: an effective amount of drospirenone; a solubilizing agent; and a permeation enhancer selected from the group consisting of alkyl lactates, carboxylic acids, alkyl esters of fatty acids, and mixtures thereof.

13. A transdermal drug delivery device according to claim 12 wherein the solubilizing agent is an aromatic solubilizing agent.

14. A transdermal drug delivery device according to claim 12 wherein the permeation enhancer comprises methyl laurate.

15. A transdermal drug delivery device according to claim 3 wherein the aromatic solubilizing agent is benzyl alcohol.

16. A transdermal drug delivery device according to claim 1 wherein drospirenone is homogenously mixed within the adhesive matrix.

17. A transdermal drug delivery device according to claim 1 wherein the adhesive matrix is substantially free of undissolved drospirenone.

18. A transdermal drug delivery device comprising: an adhesive matrix comprising: an effective amount of drospirenone; and a permeation enhancer; wherein the concentration of drospirenone in the matrix is above about 5% by weight of the total weight of the matrix and wherein the matrix is substantially free of undissolved drospirenone.

19. A transdermal drug delivery device according to claim 18 wherein the amount of drospirenone is between about 5 and 15% by weight of the total weight of the matrix.

20. A transdermal drug delivery device according to claim 18 wherein the amount of permeation enhancer is between about 5 and 30% by weight of the total of the total weight of the matrix.

21. A transdermal drug delivery device according to claim 18 and further comprising an oligomeric adjuvant selected from an oligolactic acid, oligolactic acid derivatives, or mixtures thereof.

22. A transdermal drug delivery device according to claim 18 and further comprising a solubilizing agent.

23. A transdermal drug delivery device according to claim 18 and further comprising an aromatic solubilizing agent.

24. A transdermal drug delivery device according to claim 18 wherein the permeation enhancer comprises an alkyl lactate, a carboxylic acid, or an alkyl ester of a fatty acid.

25. A transdermal drug delivery device according to claim 24 wherein the permeation enhancer comprises methyl laurate.

26. A transdermal drug delivery device according to claim 5 wherein the permeation enhancer comprises a hydroxy acid.

27. A transdermal drug delivery device according to claim 26 wherein the permeation enhancer comprises an alpha-hydroxy acid.

28. A transdermal drug delivery device according to claim 27 wherein the alpha-hydroxy acid is citric acid.

29. A transdermal drug delivery device according to claim 5 wherein the permeation enhancer comprises benzoic acid.

30. A transdermal drug delivery device according to claim 1 and further comprising an estrogen.

31. A transdermal drug delivery device according to claim 30 wherein the estrogen is ethinyl estradiol.

32. A transdermal drug delivery device according to claim 1 wherein the adhesive matrix comprises an acrylate polymer.

33. A transdermal drug delivery device according to claim 1 wherein the device provides a sustained delivery such that the transdermal flux of drospirenone at the end of use is greater than or equal to 50% of the peak flux of drospirenone during the wear period of the device.

34. A transdermal product comprising drospirenone and optionally an estrogen according to claim 1.

35. A method of transdermal delivery comprising the steps of: (a) providing a transdermal drug delivery system according to claim 1; (b) placing the delivery system in a delivering relationship to the skin of a mammal; and (c) delivering drug to the mammal.

36. A method of providing contraception to a human female comprising the steps of: (a) providing a transdermal drug delivery system having a total skin-contacting surface area of no more than 25 cm<2> and comprising at least about 20 mg of dissolved drospirenone within a pressure sensitive adhesive matrix; (b) placing the delivery system in a delivering relationship to the skin of a human female; and (c) delivering an amount of about 1 to 3 mg/day of drospirenone to the female for a period of about 7 days.

37. A method according to claim 36 wherein the pressure sensitive adhesive matrix is substantially free of undissolved drospirenone.

38. A method according to claim 35 wherein the device comprises ethinyl estradiol and further comprises the step of delivering ethinyl estradiol in an amount of about 0.01 to 0.03 mg/day to the female for a period of about 7 days.

39. A method according to claim 35 wherein the transdermal flux of drospirenone at the end of use is greater than or equal to 50% of the peak flux of drospirenone during the wear period of the device.

40. A method according to claim 36 wherein steps (a) to (c) are repeated at least 3 times, so as to provide at least 21 days of active administration without interruption.

41. A method according to claim 40 wherein a period of non-administration of at least one day follows the period of 21 days of active administration.
